(19) United States
(12) Patent Application Publication
(10) Pub. No.: US 2011/0306847 A1
(43) Pub. Date: Dec. 15, 2011

(54) IMPROVEMENTS IN THE ANALYSIS OF NEURONAL ACTIVITY

(76) Inventors: John Patrick Lowry, County Kildare (IE); Nicola Ruth Sibson, Oxford (GB)

(21) Appl. No.: 13/120,520

(22) PCT Filed: Sep. 16, 2009
(86) PCT No.: PCT/GB2009/002224

§ 371 Date: Mar. 23, 2011

(30) Foreign Application Priority Data
Oct. 8, 2008 (GB) 0818458.2

(51) Int. Cl. A61B 5/00

(52) U.S. Cl. 600/301

(57) ABSTRACT

Techniques for combining electrochemical measurements of the brain or spinal cord by voltammetry together with a scan such as magnetic resonance imaging or spectroscopy, e.g. fMRI. The techniques use particular microelectrodes, such as carbon fibre or carbon paste electrodes which do not affect the magnetic resonance measurements. The techniques allow the correlation of voltammetry and magnetic resonance measurements which in turn allows one to be used for substitution of the other in appropriate circumstances and also allows the translation of results in animal models to the human model.

The present invention relates to improvements in the analysis of neuronal activity, and in particular to measuring and correlating neurochemical, haemodynamic and metabolic processes in the human or animal brain.

### BACKGROUND

Over recent years, significant advances have been made in scanning techniques allowing imaging or spectroscopy of the human or animal brain. These include techniques based on magnetic resonance (MR) imaging and spectroscopy, PET and so on. Such scanning techniques have the advantages of being non-invasive (or relatively so), and having good spatial extent and resolution. Some techniques allow the measurement and imaging of neural activity in the brain or spinal cord of humans or other animals, allowing investigation of how the brain functions, and also of disfunction. For example functional magnetic resonance imaging (fMRI) measures the haemodynamic response related to neural activity in the brain or spinal cord. It has been known since the 1890s that changes in blood flow and blood oxygenation in the brain (collectively known as haemodynamics) are closely linked to neural activity. When nerve cells are active they consume oxygen carried by haemoglobin in red blood cells from local capillaries. The local response to this oxygen utilisation is an increase in blood flow to regions of increased neural activity, occurring after a delay of approximately 1 to 5 seconds. This leads to local changes in the relative concentration of oxyhaemoglobin and deoxyhaemoglobin, and changes in local cerebral blood volume and local cerebral blood flow (CBF). Because haemoglobin is diamagnetic when oxygenated but paramagnetic when deoxygenated, the MR signal of blood is different depending on the level of oxygenation. Appropriate MR pulse sequences have been developed to provide an image whose contrast varies depending on the blood oxygen level (blood-oxygen level dependent (BOLD) contrast). It should be noted that the blood oxygen level can change positively or negatively depending on relative changes in the cerebral blood flow and oxygen consumption. Thus increases in CBF that outstrip changes in oxygen consumption will lead to an increased BOLD signal intensity, whereas decreases in CBF which are outstripped by oxygen consumption will cause decreased BOLD signal intensity.

Although fMRI has proved of great interest, in particular because changes in blood oxygen level in parts of the brain can be imaged while the subject is performing a cognitive task, such measurements do not provide a full understanding of brain function because they do not directly measure neuronal activity, but only the haemodynamics of local regions, and in particular only the relationship between oxygen consumption and supply. Thus although such techniques are being used to evaluate neurological conditions such as multiple sclerosis, stroke and Alzheimer's disease, this relies on the assumption that alterations in neuronal activity resulting in alterations in the MR measurements are indicative of processes such as neuronal disfunction and neuronal plasticity. Further, fMRI responses are often compared across multiple brain regions assuming a constant relationship between neuronal, haemodynamic and metabolic processes but such assumptions are not always valid.

### SUMMARY OF THE INVENTION

The present invention provides a way of correlating the results of scanning techniques such as MR imaging and spectroscopy or PET with measurements of neurochemical changes that are much more closely related to neuronal activation. This is achieved by simultaneously performing the scan while making neurochemical measurements on the same part of the subject brain, and correlating the results. The neurochemical measurements are performed by voltammetric techniques using implanted amperometric electrodes.

Voltammetric measurements are obtained by implanting electrodes into the subject's brain, usually at least three electrodes—a working electrode, reference electrode and auxiliary electrode though four or more may be used, depending on the number of working electrodes required, applying an electrical potential to the electrodes and measuring the current between the working and auxiliary electrode. The current depends on the presence of electroactive analytes at the surface of the working electrode. The applied electric potential can be steady, or more usually, varied with time in a predetermined profile. The electrodes can either detect electroactive analytes directly (for analytes in which electron transfer takes place directly to the electrode surface), or can be biosensors which involve catalysis of the electron transfer, usually by means of an analyte-specific enzyme immobilised on the electrode. Voltammetric techniques are described in the paper Neuroanalytical Chemistry in vivo Using Electrochemical Sensors by John P Lowry and Robert D O'Neill, Encyclopeadia of Sensors, Volume X: pages 1-23; 2000, which is hereby incorporated by reference.

The advantage of voltammetric measurements are that the temporal and spatial resolution are high: of the order of millisecond and micron respectively. Thus they provide very specific information about the neurochemistry of the specific part of the brain where the working electrode is implanted. Correlating this specific neurochemical information with the results of a simultaneous scan allows improved understanding of how brain function relates to specific neuronal activation.

An additional advantage of correlating scan results, such as magnetic resonance measurements which measure haemodynamics, with voltammetric measurements (which measure neurochemistry) is that it allows a translation between the two techniques and between human and animal models based on measurements using only one of the techniques. Thus, using fMRI as an example of a scanning technique, the correlated data sets allow new voltammetric measurements to be used to estimate or predict the results of an fMRI scan without the need to perform such a scan. Further, knowledge of haemodynamic processes in a human brain which correspond to haemodynamic processes in an animal brain, and how haemodynamic processes in a human brain relate to human cognitive processes, allow voltammetric measurements made in an animal to predict the effect on cognitive processes in a human. Such prediction is useful by allowing the testing of potentially pharmacologically active substances in an animal to estimate their effects on human neuronal disorders.

Accordingly a first aspect of the invention provides a method comprising the steps of performing scan measurements on a region within a subject's brain or spinal cord to collect scan data over a predetermined time period; performing voltammetric measurements at a position within said region during said time period; correlating said scan data with said voltammetric measurements and outputting said correlated measurements.

Another aspect of the invention provides a method comprising the steps of performing scan measurements on a region within a subject's brain or spinal cord to collect scan data over a predetermined time period; correlating said scan data with a set of voltammetric measurements relating to a position within said region during said time period and outputting said correlated measurements.

A further aspect of the invention provides a method of neurochemical analysis of a subject's brain or spinal cord using a previously obtained first data set comprising scan data from a region within a subject's brain or spinal cord over a predetermined time period, and a second data set comprising voltammetric measurements obtained from a position within said region during said time period, the method comprising: reading said first data set and said second data set, correlating said scan data with said voltammetric measurements, and outputting said correlated measurements.

A further aspect of the invention provides a method of estimating the effect on the human brain of pharmacological intervention by administration of a pharmacologically active substance to a human, comprising performing voltammetric measurements at a position within a region of an animal subject's brain during a predetermined time period following administration of the pharmacologically active substance to the animal; comparing the obtained voltammetric measurements to correlated scan data and voltammetric measurements obtained by the method of claim 1, 2 or 3 to estimate the haemodynamic response of the animal brain to said pharmacological intervention, obtaining scan data linking haemodynamic and cognitive processes in a human brain; and comparing the estimated haemodynamic response of the animal brain to the haemodynamic processes in the human brain to find corresponding processes to estimate the effect on the human brain of pharmacological intervention by administration of the pharmacologically active substance to a human.

With the invention the correlated measurements may be displayed to indicate their temporal relationship.

The scan measurements may be magnetic resonance images or spectroscopy, such as fMRI, diffusion-weighted imaging or carbon 13 spectroscopy, or other scanning techniques such as PET, SPECT, in-vivo microscopy, etc.

The voltammetric measurements may be obtained using carbon-based electrodes, e.g. carbon fibre or carbon paste electrodes, optionally treated to be analyte-specific, for example to provide analyte-specific enzyme catalysis at the electrode surface. Alternatively other non-magnetic electrode materials e.g. platinum or semiconductor electrodes may be used. The electrodes may incorporate an oxygen reservoir for releasing oxygen to feed to the catalytic biosensor process.

Electrodes suitable for use in the invention are known to those in the art and are described in the papers mentioned above and below, incorporated herein by reference.

### LIST OF DRAWINGS

The invention will be further described by way of example with reference to the accompanying drawings in which:

FIG. 1 schematically illustrates the simultaneous recording of fMRI and voltammetry data;

FIG. 2 shows the results of simultaneous measurement of brain tissue oxygen using a carbon fiber electrode and the BOLD fMRI signal in a rat cortex in response to brief (see A. two minutes) period of increased oxygen concentrations (hashed lines);

FIG. 3 schematically illustrates the concept of preclinical to clinical translation of pharmacological intervention using magnetic resonance measurements on a human subject and magnetic resonance and voltammetry measurements on an animal subject;

FIG. 4 schematically illustrates the use of voltammetry measurements together with collated voltammetry and cerebral blood flow data from fMRI such that voltammetry measurements can be used to estimate corresponding cerebral blood flow;

FIG. 5 illustrates schematically an example of a carbon fibre electrode;

FIG. 6 illustrates a multiecho gradient echo image acquired in the coronal plane through a rat head with a carbon fibre electrode implanted in the brain.

### DESCRIPTION OF SPECIFIC EMBODIMENTS

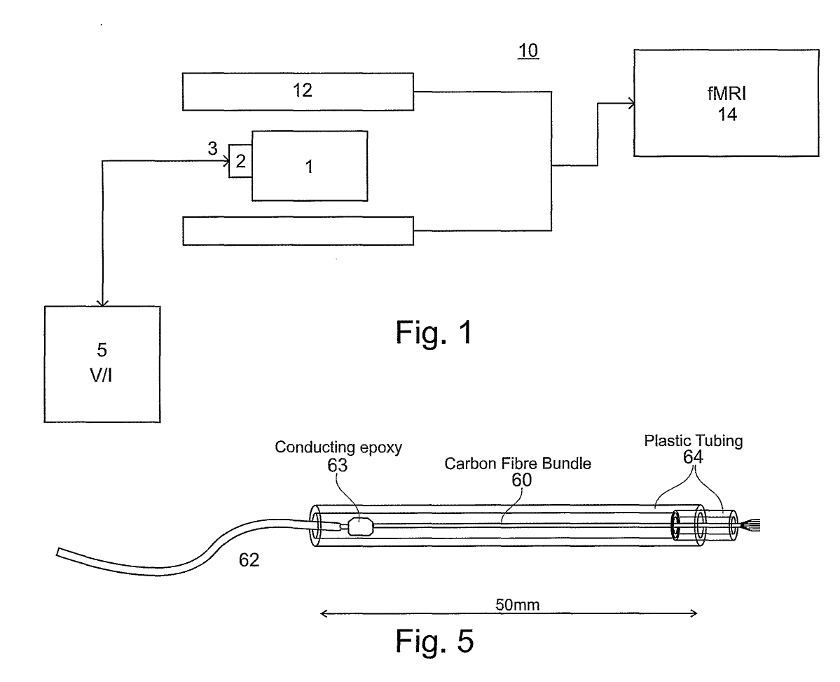

As illustrated in FIG. 1 a first embodiment of the invention provides for the correlation of brain or spinal cord scan results, in this example magnetic resonance measurements on the brain, with electrochemical measurements made using amperometric electrodes and voltammetric techniques. As illustrated in FIG. 1 by implanting a microvoltammetric working electrode 3 in the brain 2 of an animal or human subject 1 with suitable reference and auxiliary electrodes as is conventional, applying a suitable electric potential profile by voltammetry controller 5, and recording in the controller 5 the resulting Faradaic current, changes in the concentration of a variety of substances in the extra-cellular fluid can be monitored with sub-second time resolution over extended periods in the subject. Thus this technique allows the detection of signalling substances released from nerve cells into the extra cellular fluid, and provides an understanding of the function and role of specific neurochemicals in neurosignalling. The working electrode 3 can be of the classical type where electron transfer from a target analyte takes place directly to the electrode surface, or can be a biosensor which involves catalysis of the electron transfer, usually by means of an analyte-specific enzyme immobilised on the electrode. Sensors are available for glucose, ascorbate and oxygen, and also other substances such as lactate, glutamate and NO, dopamine, D-Serine, blood flow, serotonin, acetylcholine, pyruvate. Techniques for performing voltammetry in vivo and for making the electrodes are known to those in the art and are described in the following papers incorporated herein by reference:

O'Neill R. D. & Lowry J. P. (2000) Voltammetry in vivo for chemical analysis of the living brain. In Encyclopaedia of Analytical Chemistry (Edited by Meyers R.), John Wiley & Sons Ltd, Chichester, pp. 676-709.

Lowry J. P. & Fillenz M. (1997) Evidence for uncoupling of oxygen and glucose utilization during neuronal activation in rat striatum. J. Physiol. (London), 498, 497-501.

Lowry, J. P., Bouteille, M. G. and Fillenz, M. (1997) Measurement of brain tissue oxygen at a carbon paste electrode can serve as an index of increases in regional cerebral blood flow, Journal of Neuroscience Methods, 71, 177-182.

For example, changes in O2 at implanted carbon fibre electrodes (CFEs) can be monitored using Constant Potential Amperometry (CPA), where the detecting electrode is held at a constant potential sufficient to detect the oxidation or reduction of the target substance. For O2 detection the CFE is held at −900 mV which is in the mass-transport limited region after the peak potential for O2 reduction. This ensures that the signal is not compromised by detection of other reducible species present in the brain. Constant potential amperometry can be carried out using a low-noise potentiostat (Biostat II, Electrochemical and Medical systems, Newbury, UK). Data acquisition is performed out with a Gateway GP6-350 computer, a Powerlab/400 interface system (ADInstruments Ltd., Oxford, UK) and Chart for Windows (v 4.0.1) software (ADInstruments Ltd.).

Although voltammetry techniques provide high spatial and temporal resolution (approximately 10 micrometers spatial and millisecond temporal resolution), the sensors are currently limited to a single analyte and, of course, they only give information about a small defined region in the brain. Thus in accordance with the invention the voltammetric measurement technique is performed simultaneously with a brain scan, in this embodiment an fMRI scan, which offers whole brain images.

The scanner 10 consists of a scan controller 14 which causes the scanning apparatus 12 to apply the necessary magnetic and radio frequency fields and sense the signals from the subject 1 in the conventional way. Techniques for performing functional magnetic resonance imaging of the brain are described in, for example, Ogawa, S., Lee, T. M., Kay, A. R. and Tank, D. W. (1990) Brain magnetic resonance imaging with contrast dependent on blood oxygenation, Proc. Natl. Acad. Sci., 87, 9868-72, but since then fMRI has become well known and is routinely available.

Magnetic resonance measurements rely on the subject being kept still in a scanner 10 in a high magnetic field. It has not, however, been possible to perform magnetic resonance measurements in combination with techniques involving the implantation of electrodes as the electrodes, and related connections and circuitry, tend to cause undesirable artefacts on the magnetic resonance measurements, and the use of magnetic materials in most MR scanners is not possible.

The inventors have found that by using carbon-based electrodes, e.g. carbon fiber or carbon paste electrodes for the voltammetry measurements, the occurrence of artefact in the scan results is avoided and this therefore allows the simultaneous obtaining of voltammetry and scans in the brain or spinal cord.

Techniques for making carbon fiber and carbon past electrodes are described in the review paper “Neuroanalytical Chemistry in vivo Using Electrical Chemical Sensors” by Lowry and O'Neill mentioned above. As explained there, to make a carbon paste electrode a mixture of carbon paste consisting of carbon powder and silicone oil together with epoxy resin is force-filled into a glass capillary (about 50-200 microns in diameter) and heat cured. A carbon fibre electrode (diameter ˜100 μm), such as that illustrated in FIG. 5 can be prepared by attaching carbon fibre threads 60 (Goodfellow, Cambridge, UK) to copper wire 62 using conductive silver epoxy 63 (Chemtronics, Georgia, USA). Fibres are encased within a double-layered plastic insulating sheath 64. The carbon fibre is exposed ca. 2.5 mm beyond the insulating sheath 64 for insertion under the skull of the subject. To prevent the carbon fibre threads splitting, the electrode tip may be coated with cyanoacrylate (Loctite Ltd, Ireland) producing a cylinder electrode, or the shaft of the wire coated with a supportive insulating layer (e.g. cyanoacrylate or a polymeric material such as polystyrene). Although carbon fibre is a non-magnetic material, the conductive wire and silver epoxy can cause significant image artefacts if positioned close to the subjects' head. To prevent this, the total length of carbon fibre threads used is sufficiently long so that the connection point lies outside the RF coil once the subject is inside the scanner.

More generally the electrodes can be prepared by sealing single or multiple strands of carbon fiber into glass, silica or Teflon capillaries and this can provide electrodes of a small diameter (5-50 microns) with a desired length from 0-50 micrometers). As explained in the review paper the electrodes can be adapted by applying coatings to improve selectivity and sensitivity to different analytes and can be used as the basis for biosensors by immobilising on the electrode a sensitive and selective biological element, such as an enzyme, plant or animal tissues, microbes and antibodies.

As indicated above BOLD fMRI methods are known, but an example used in an embodiment of the present invention consists of a multi-echo gradient echo imaging sequence used to minimize artifacts arising from the presence of electrodes in the brain, with the following parameters: flip angle=20°; relaxation time (TR)=27.3 ms; echo time (TE)=7, 14, 21 ms (acquired within one TR); acquisition matrix=192×64 (zero filled to 192×96); 1 slice, voxel size 0.47 mm×0.47 mm×1.5 mm; single average (dc offset corrected offline); 1.75 s per acquisition. Following data acquisition, mean echo images are calculated from the arithmetic mean of the individual echo images. Subsequently, the brain is manually masked to remove extra-cranial signal and images are Gaussian smoothed with a kernel 2 times the in-plane resolution. A semi-model free approach IRVA (Inter-Repetition Variance Analysis) is used to determine repeated epoch specific variances. Regions of interest (ROIs) are delineated on the images and time-courses of signal response are obtained for each ROI from the mean echo data.

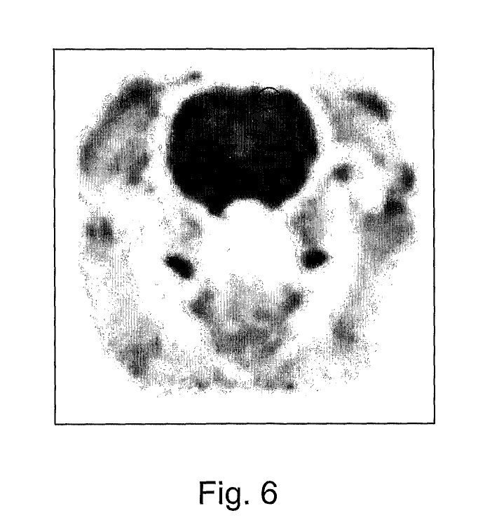

FIG. 6 illustrates a multiecho gradient echo image acquired at 7 Tesla in the coronal plane Through a rat head. The brain can be seen in the dorsal half of the head, and little artefact from the carbon fibre electrode (right cortex—circle indicates site of electrode) is evident.

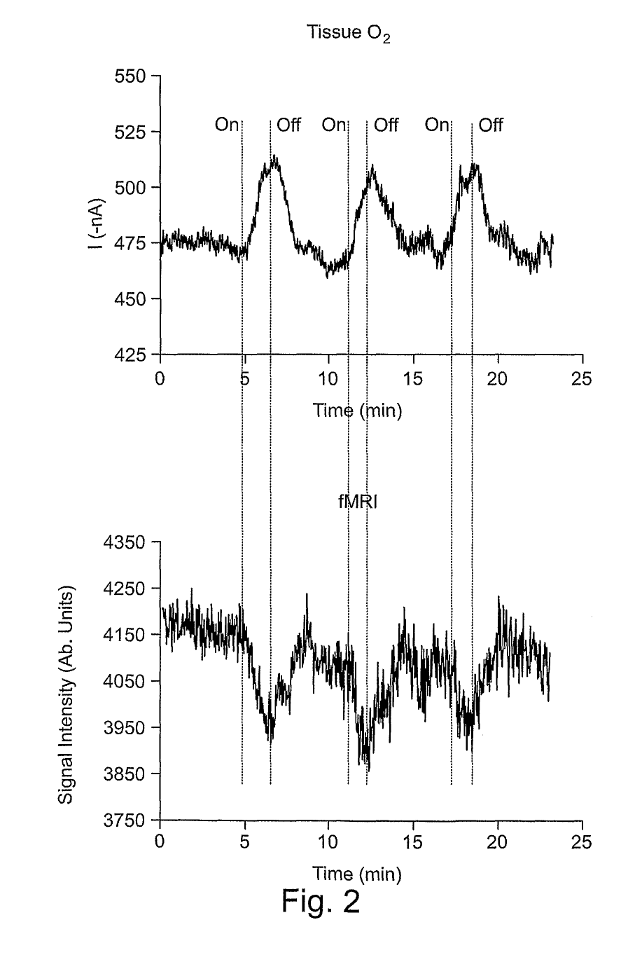

FIG. 2 illustrates the results of performing simultaneous measurement of brain tissue oxygen using a carbon fiber electrode together with the BOLD fMRI signal from the region (voxel) corresponding to the working electrode position in a rat cortex in response to brief periods of increased oxygen concentrations (indicated by the hashed lines). As can be seen both the voltammetry and magnetic resonance measurements show a periodic variation in response to the periods of increased oxygen concentrations. Thus by correlating the signals from the magnetic resonance measurements and voltammetry measurements both temporally and spatially and, for example, by displaying them together in alignment on a time axis, it is possible to link neurochemical changes in the extracellular fluid with haemodynamic activity in the brain.

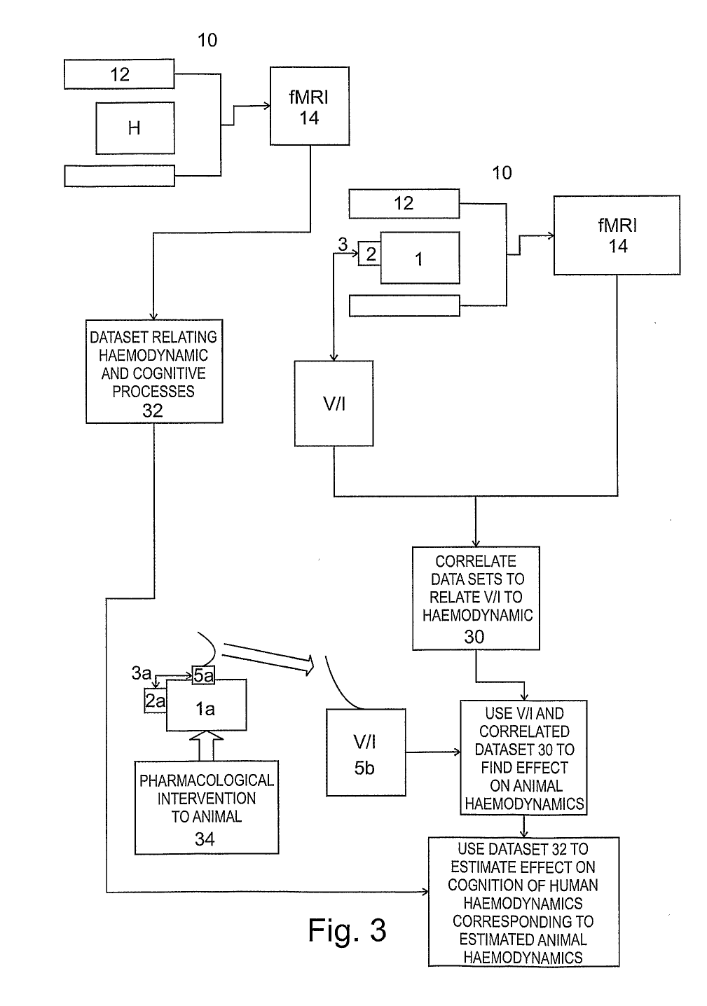

FIG. 3 of the accompanying drawings illustrate schematically how the correlation between scan measurements and voltammetry measurements can be used to advantage in understanding how pharmalogical substances affect brain function. In particular one of the major hurdles in the discovery of new medicines to treat psychiatric and neurological disorders is the paucity of suitable animal models capable of predicted clinical benefit (i.e. benefit in humans). This is particularly true of disorders associated with cognitive disturbance such as schizophrenia and Alzheimer's disease. It would be useful to have techniques for translating from human to animal models in the measurement of cognition, and to have ways of monitoring neuronal activation in a freely moving and behaving animal in a way that can be correlated with cognitive performance and its interaction with drugs. FIG. 3 illustrates schematically how the invention can assist in these aims, again using fMRI as the example scanning technique.

The first step is to obtain the correlation between voltammetry measurements and magnetic resonance measurements, for example by performing simultaneous magnetic resonance measurements using a scanner such as an fMRI scanner 10 and voltammetry measurements using an implanted electrode 3 in an animal subject 1. This produces correlated data sets 30 which relate the neurochemical measurements resulting from voltammetry with the haemodynamic measurements from fMRI.

It is also possible to obtain a data set 32 which relates haemodynamic data to cognitive processes in a human subject H by performing an fMRI scan while giving the subject H cognitive tasks.

In order to test the effect of pharmacological intervention on brain function, an animal subject 3a can be administered with the pharmacological substance as indicated at 34 while voltammetry measurements are made using an implanted electrode 3a and controller 5a. As schematically illustrated in FIG. 3 it is possible by making the controller in the form of a wireless module carried by the animal that these measurements can be made while the animal is freely moving and behaving. The measurements can be transmitted by the wireless controller 5a to a stationary station 5b where the measurements are then stored. Then, by using the correlated data sets 30, the effect that this pharmacological intervention would be expected to have on the haemodynamics in the animal brain is estimated from the voltammetry measurements on the animal subject 3a taken during pharmacological intervention. Then using the data set 32 it is possible to estimate the effect that similar haemodynamic changes would have on cognitive processes in the human.

Thus although this aspect of the invention does not provide direct measurement on a human model of the effect on cognition of pharmacological intervention, it provides a translational bridge between the human and animal models which is useful in pre-clinical neuropharmacological and behavioural investigations associated with drug discovery.

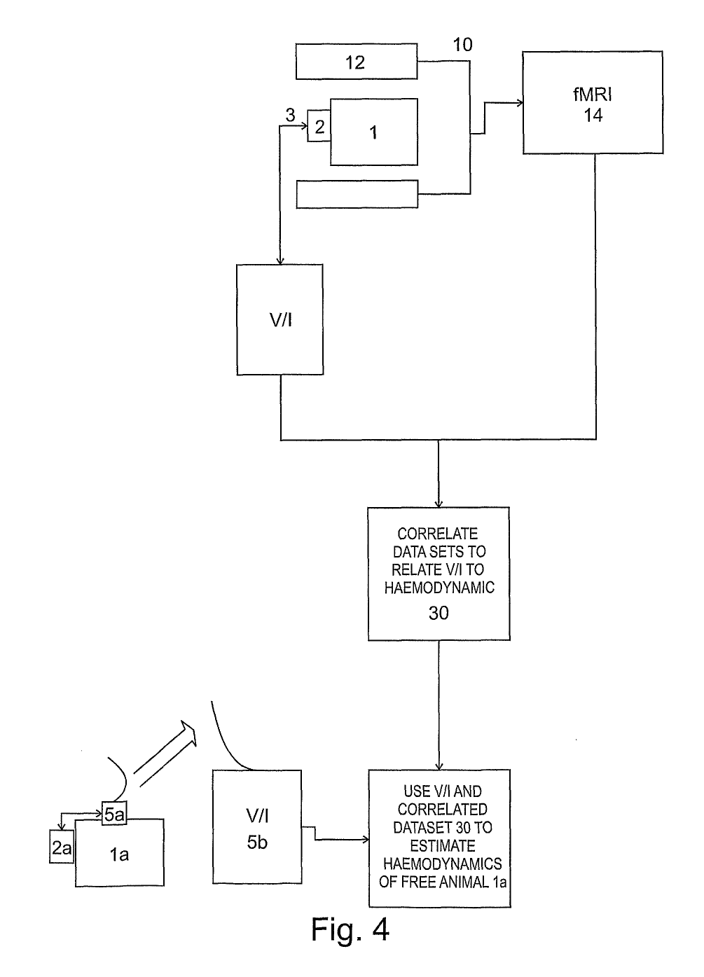

The correlation of voltammetry and magnetic resonance measurements also allows one technique to be used in substitution for the other, e.g. in situations where one or the other is not appropriate. For example, an advantage of voltammetry is that measurements can be made on a freely moving and behaving animal as mentioned above by using an implanted electrode 3a controlled by a wireless controller 5a in communication with a central station 5b. FIG. 4 illustrates how voltammetry can therefore be used to estimate haemodynamics without direct haemodynamic measurement.

Firstly it is necessary to prepare correlated data sets of voltammetry and magnetic resonance measurements 30 by simultaneous performance of the magnetic resonance measurements using scanner 10 and voltammetry using the implanted electrode 3 and controller 5. Then, as described above, voltammetry measurements can be made on a freely behaving animal 1a and the result used together with the correlated data set 30 to estimate the haemodynamics in the animal's brain. It will be appreciated that brain and spinal cord scans can only be made on a subject which is kept still within the scanner space, so that it is not possible directly to measure, for example, brain haemodynamics of a freely moving and behaving subject. Further, scanning tends to be expensive. The use of the correlated data sets 30 allows voltammetry to act as a cheaper substitute for scan techniques such as fMRI in some circumstances, but also adds the advantages of high, spatial and temporal resolution and avoiding restriction on the subject.

1. A method comprising the steps of performing imaging measurements on a region within a subject's brain or spinal cord to collect image data over a predetermined time period; performing voltammetric measurements at a position within said region during said time period; correlating said image data with said voltammetric measurements and outputting said correlated measurements.

2. A method comprising the steps of: performing imaging measurements on a region within a subject's brain or spinal cord to collect image data over a predetermined time period; correlating said image data with a set of voltammetric measurements relating to a position within said region during said time period and outputting said correlated measurements.

3. A method of neurochemical analysis of a subject's brain or spinal cord using a previously obtained first data set comprising image data from a region within a subject's brain or spinal cord over a predetermined time period, and a second data set comprising voltammetric measurements obtained from a position within said region during said time period, the method comprising: reading said first data set and said second data set, correlating said scan data with said voltammetric measurements, and outputting said correlated measurements.

4. A method of estimating the effect on the human brain of pharmacological intervention by administration of a pharmacologically active substance to a human, comprising performing voltammetric measurements at a position within a region of an animal subject's brain during a predetermined time period following administration of the pharmacologically active substance to the animal; comparing the obtained voltammetric measurements to correlated image data and voltammetric measurements obtained by the method of claim 1, 2 or 3 to estimate the haemodynamic response of the animal brain to said pharmacological intervention, obtaining image data linking haemodynamic and cognitive processes in a human brain; and comparing the estimated haemodynamic response of the animal brain to the haemodynamic processes in the human brain to find corresponding processes to estimate the effect on the human brain of pharmacological intervention by administration of the pharmacologically active substance to a human.

5. A method according to any one of the preceding claims wherein the step of outputting said correlated measurements comprises displaying the voltammetric measurements and magnetic resonance measurements and their temporal relationship.

6. A method according to claim 5 wherein said correlating is temporal and spatial.

7. A method according to any one of the preceding claims wherein the imaging measurements are magnetic resonance measurements.

8. A method according to claim 8 wherein said imaging measurements comprise fMRI.

9. A method according to claim 7 wherein performing said magnetic resonance measurements comprises diffusion-weighted imaging.

10. A method according to any one of the preceding claims wherein said voltammetry measurements uses carbon-based electrodes, e.g. carbon fibre or carbon paste electrodes.

11. A method according to any one of claims 1 to 10 wherein said voltammetry measurements use non-magnetic electrodes.

12. A method according to claim 10 or 11 wherein the working electrode is treated with biorecognition element, e.g. an enzyme-catalyst.

13. A method according to claim 10, 11 or 12 wherein the working electrode incorporates an oxygen reservoir.
